(19) United States
(12) Patent Application Publication
(10) Pub. No.: US 2019/0311810 A1
(43) Pub. Date: Oct. 10, 2019

(54) SYSTEM AND METHOD FOR FACILITATING COMPUTATIONAL ANALYSIS OF A HEALTH CONDITION

(71) Applicant: KONINKLIJKE PHILIPS N.V., EINDHOVEN (NL)

(72) Inventors: Merlijn SEVENSTER, Haarlem (NL); Thomas Andre FORSBERG, Hayward, CA (US); Wilhelmus Johannes Allegonda Franciscus DIRKS, Deurne (NL)

(73) Assignee: KONINKLIJKE PHILIPS N.V., EINDHOVEN (NL)

(21) Appl. No.: 16/467,067

(22) PCT Filed: Dec. 12, 2017
(86) PCT No.: PCT/EP2017/082492

§ 371 Date: Jun. 6, 2019

Related U.S. Application Data
(60) Provisional application No. 62/432,721, filed on Dec. 12, 2016.

(51) Int. Cl. G16H 50/30; G06F 16/901; G16H 10/60

(52) U.S. Cl. CPC G16H 50/30; G16H 10/60; G06F 16/9024

(57) ABSTRACT

The present disclosure pertains to a system configured to facilitate computational analysis of a health condition. In some embodiments, the system is configured to: obtain a graph comprising nodes and edges, the nodes comprising nodes of a first node type that correspond to risk parameters and nodes of a second node type that correspond to risk models; process the graph to generate a resulting graph for a first individual by: determining a value of a risk parameter of a first-type node (that has an edge linking the first-type node to a second-type node in the graph) with respect to the first individual; and removing edges linking the second-type node to first-type nodes from the graph based on the value of the risk parameter of the first-type node; and select, based on the resulting graph, risk models to be used to perform analysis of the first individual's health condition.

### BACKGROUND

### 1. Field

The present disclosure pertains to a system configured to facilitate computational analysis of a health condition.

### 2. Description of the Related Art

Computer-assisted health assessment systems enable physicians, other medical personnel, or other users to more quickly and accurately assess an individual's health risk or determine other information about the individual. These health assessment systems typically rely on risk models to facilitate such assessment. As the number of risk models supported by health assessment systems continue to grow, however, so does the number of underlying risk parameters (e.g., which the risk models take as input parameters) along with the requirement of such health assessment systems to manage the large number of risk models and risk parameters. As an example, the large number of risk models and risk parameters may not only increase the burden on a user to confirm risk factors, risk markers, or other risk parameters, but may also waste computing resources in executing one or more irrelevant risk models.

### SUMMARY

Accordingly, one or more aspects of the present disclosure relate to a system configured to facilitate computational analysis of a health condition. The system comprises one or more hardware processors and/or other components. In some embodiments, the one or more hardware processors are configured by machine readable instructions to: obtain a graph comprising nodes and edges, each of the edges linking two of the nodes, the nodes comprising nodes of a first node type that respectively correspond to risk parameters and nodes of a second node type that respectively correspond to risk models, the risk models being configured to take one or more values of the risk parameters as input to estimate a likelihood that an individual has or is at risk of having one or more health conditions; process the obtained graph to generate a resulting graph for a first individual, wherein processing the obtained graph comprises: determining one of the first-type nodes as a node to be assessed, the first-type node having an edge linking the first-type node to a second type node in the obtained graph; determining a value of a risk parameter of the first-type node with respect to the first individual; and removing one or more edges linking the second-type node to one or more first-type nodes, including the edge linking the first-type node and the second-type node, from the obtained graph based on the value of the risk parameter of the first-type node; and select, based on the resulting graph, one or more risk models to be used to perform analysis of at least one health condition of the first individual such that the one or more risk models are selected from a set of risk models corresponding to one or more second-type nodes of the resulting graph that respectively have at least one edge linking the respective second-type node to at least one first-type node of the resulting graph.

Yet another aspect of the present disclosure relates to a method for facilitating computational analysis of a health condition. The method is implemented by one or more hardware processors configured by machine readable instructions and/or other components. In some embodiments, the method comprises: obtaining a graph comprising nodes and edges, each of the edges linking two of the nodes, the nodes comprising nodes of a first node type that respectively correspond to risk parameters and nodes of a second node type that respectively correspond to risk models, the risk models being configured to take one or more values of the risk parameters as input to estimate a likelihood that an individual has or is at risk of having one or more health conditions; processing the obtained graph to generate a resulting graph for a first individual, wherein processing the obtained graph comprises: determining one of the first-type nodes as a node to be assessed, the first-type node having an edge linking the first-type node to a second type node in the obtained graph; determining a value of a risk parameter of the first-type node with respect to the first individual; and removing one or more edges linking the second-type node to one or more first-type nodes, including the edge linking the first-type node and the second-type node, from the obtained graph based on the value of the risk parameter of the first-type node; and selecting, based on the resulting graph, one or more risk models to be used to perform analysis of at least one health condition of the first individual such that the one or more risk models are selected from a set of risk models corresponding to one or more second-type nodes of the resulting graph that respectively have at least one edge linking the respective second-type node to at least one first-type node of the resulting graph.

Still another aspect of the present disclosure relates to a system for facilitating computational analysis of a health condition. In some embodiments, the system comprises: means for obtaining a graph comprising nodes and edges, each of the edges linking two of the nodes, the nodes comprising nodes of a first node type that respectively correspond to risk parameters and nodes of a second node type that respectively correspond to risk models, the risk models being configured to take one or more values of the risk parameters as input to estimate a likelihood that an individual has or is at risk of having one or more health conditions; means for processing the obtained graph to generate a resulting graph for a first individual, wherein processing the obtained graph comprises: determining one of the first-type nodes as a node to be assessed, the first-type node having an edge linking the first-type node to a second type node in the obtained graph; determining a value of a risk parameter of the first-type node with respect to the first individual; and removing one or more edges linking the second-type node to one or more first-type nodes, including the edge linking the first-type node and the second-type node, from the obtained graph based on the value of the risk parameter of the first-type node; and means for selecting, based on the resulting graph, one or more risk models to be used to perform analysis of at least one health condition of the first individual such that the one or more risk models are selected from a set of risk models corresponding to one or more second-type nodes of the resulting graph that respectively have at least one edge linking the respective second-type node to at least one first-type node of the resulting graph.

These and other objects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

FIG. 1 is a schematic illustration of a system configured to facilitate computational analysis of a health condition, in accordance with one or more embodiments.

FIGS. 2A, 2B, and 2C illustrate examples of relevant and irrelevant risk parameters in respective tables together with their corresponding status values that relate to a particular individual, in accordance with one or more embodiments.

FIGS. 3A, 3B, 3C, 3D, and 3E illustrate examples of risk model nodes in a graph connected via edges to risk parameter nodes, in accordance with one or more embodiments.

FIG. 4 illustrates a method for facilitating computational analysis of a health condition via graph generation, in accordance with one or more embodiments.

### DETAILED DESCRIPTION OF EXEMPLARY EMBODIMENTS

As used herein, the singular form of “a”, “an”, and “the” include plural references unless the context clearly dictates otherwise. As used herein, the term “or” means “and/or” unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

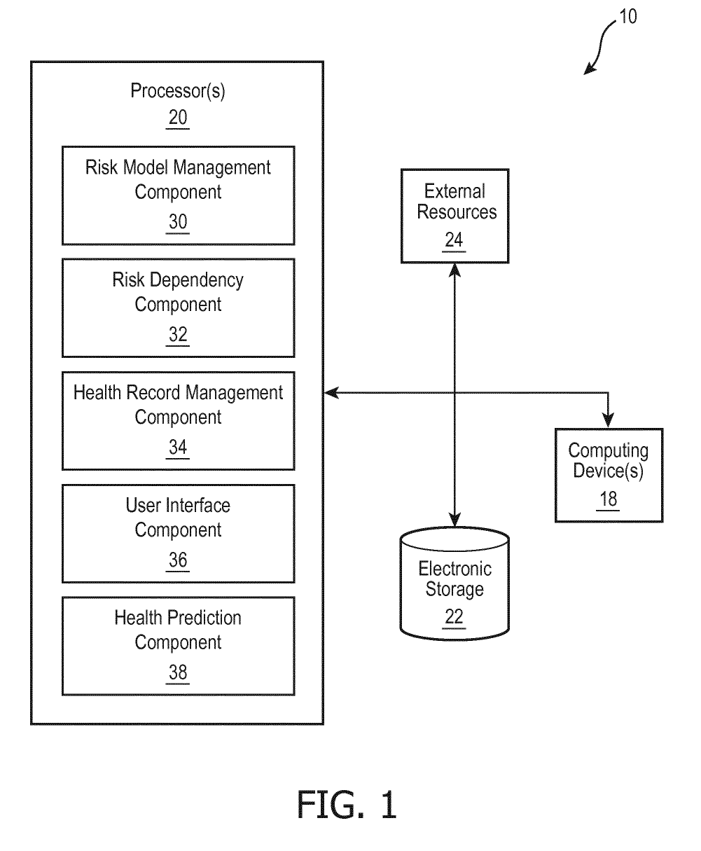

FIG. 1 illustrates a system 10 configured to facilitate computational analysis of a health condition, in accordance with one or more embodiments. System 10 may be configured to help users of the system confirm whether an individual (e.g., a patient or other individual) associated with certain, extracted health data has or is likely to have a risk parameter (e.g., risk factor, risk marker, or other risk parameter). A risk factor may be a variable associated with an increased risk of disease or infection, and a risk marker may be a variable that is quantitatively associated with a disease or other outcome. System 10 may identify potentially relevant risk parameters for a user to confirm and/or automatically confirm the relevancy thereof.

Risk parameters may serve as inputs to risk models, which may be run to predict the likelihood that an individual has or is at risk of having one or more health conditions (e.g., a disease, a clinical condition, or other adverse health-related state). As the number of risk parameters grows, so does the number of risk models that could be run to make predictions or determine probabilities. Execution of each risk model may be computationally intensive and time consuming, which may be unacceptable to users needing immediate predicted/probabilistic outcomes. In some embodiments, among other benefits, system 10 may resolve the need by identifying risk parameters that could have their relevancy confirmed to then narrow the number of risk models to be run.

Disclosed embodiments facilitate a user in confirming, establishing, or assessing risk parameters and determining outcomes (e.g., risk scores or other outcomes) as a result of running risk models. Additionally, some embodiments account for dependencies between the risk parameters and risk models, and they minimize both the number of risk parameters that need confirmation by the user and the number of risk models that need to be run. Removal of irrelevant tasking from a medical worker's agenda may result in more efficient use of time and improved quality of outcomes, e.g., by not distracting the medical worker with risk parameters and risk models that are known to be unhelpful or irrelevant.

In some embodiments, system 10 may use a graphical representation of risk models with their relationship to risk parameters (e.g., a graphical representation of the risk parameters, risk models, and dependencies thereof). The graph may learn or leverage relationships between the growing number of risk parameters and risk models. For example, there may be hundreds, thousands, or millions of risk parameters and hundreds, thousands, or millions of risk models. Each risk model may take value(s) of one or more risk parameters as input, and the risk parameters themselves may be determined (e.g., synthesized or generated) by system 10. System 10 may determine the risk parameters by extracting relevant health data from one or more sources of medical information.

In some embodiments, system 10 may predict a set of relevant risk parameters. The predicted set of risk parameters may be presented to a user of system 10 for relevance confirmation. A user (e.g., a nurse, doctor, medical worker, or other personnel) may confirm the presence or risk of a given risk parameter on a user interface. For example, the user may confirm whether the risk parameter is relevant, or the user may establish another characteristic of the risk parameter. One aspect of the present disclosure is therefore to assist users of system 10 to determine and confirm risk parameters.

As shown in FIG. 1, system 10 may provide interfaces to and from external resources 24, electronic storage 22, or another database. System 10 may have access to medical information, such as from hospital information systems (HIS), clinical data repositories (CDR), Electronic Medical Records (EMR), and other sources. The collected medical information may include useful health data and patient information, such as demographic or background information, of an individual. System 10 may analyze the medical information and accordingly predict risk parameters.

Accessing and processing the medical information is often inefficient.

Some embodiments improve on past systems by tailoring the medical information by context (e.g., for a particular physician). For instance, a radiologist interpreting a computed tomography (CT) study for an abdomen may seek to determine whether each of risk parameters A, B, and C is present but not risk parameters X, Y, and Z. System 10 may filter out risk parameters X, Y, and Z or demote their importance (e.g., in an ordering of risk parameters) in a presentation to the user. System 10 may perform this filtering of risk parameters based on health data (e.g., of the individual) extracted from the medical information.

A risk parameter is a potentially composite construction that is defined in terms of health data, including in some instances a plurality of health data points. In some embodiments, health data is shared between risk parameters. Health data may have a hierarchical relationship, when embedded ontologically. A disease state or disease profile may be a combination of one or more risk parameters. Example risk parameters may pertain to an individual's age, the individual's gender, whether the visit to the doctor is due to an emergency, or other health related parameters. Other example risk parameters pertain to a disease state of the individual (e.g., a likelihood of having a clinical condition or a risk for having the clinical condition).

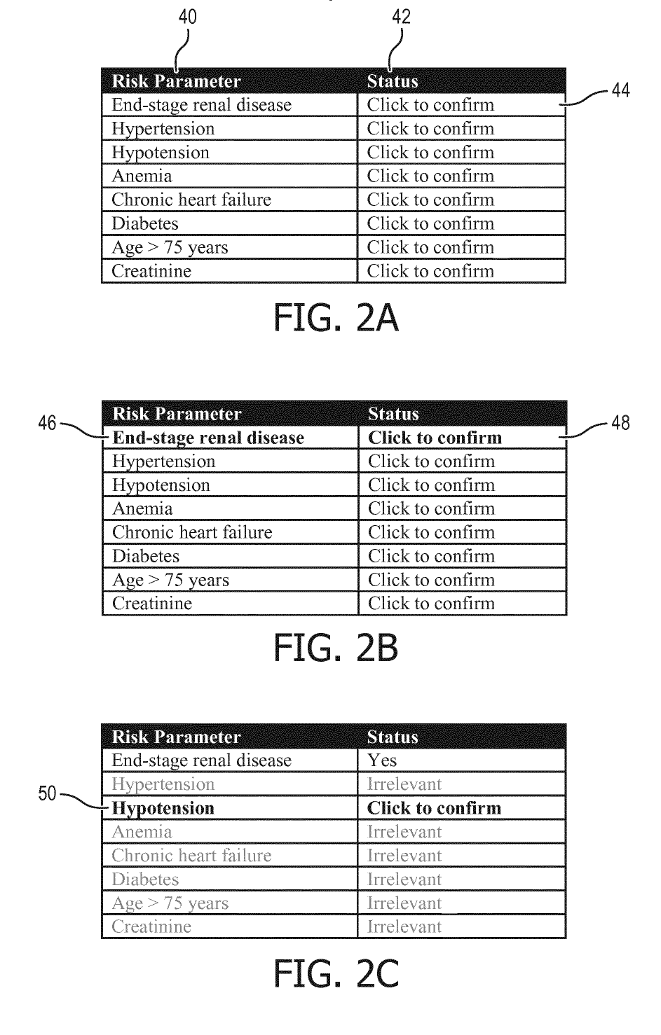

FIG. 2A illustrates in a table several example risk parameters that may be presented to a user on a user interface of system 10 such that the user may confirm one or more of the risk parameters. The table may comprise a column of risk parameters 40 and a corresponding status column 42. The user may make the confirmation(s) on the user interface, e.g., by clicking the “Click to confirm” hyperlink or button 44. Since there may be many potentially relevant risk parameters, the risk parameters shown in FIG. 2A may, in some embodiments, be in an ordering. In some embodiments, though, the user may confirm a risk parameter without the risk parameter ordering or another form of guidance for the user.

In some embodiments, the number of irrelevant risk parameters that are accidentally or erroneously confirmed as relevant drops significantly when system 10 predicts relevant risk parameters, as opposed to the user having to confirm all risk parameters that need confirmation. System 10 therefore implements a form of a failsafe to help preclude users from confirming irrelevant risk parameters. Also, system 10 may implement a manner for confirming risk parameters as relevant in a more efficient manner (e.g., by not flooding the user with risk parameters known to be irrelevant from experience or from prior confirmation).

System 10 may predict risk parameters for patients with limited medical information. For example, in some embodiments, system 10 self-learns decision criteria for predicting risk parameters based not only on the medical information but also on user-system interactions (e.g., prior confirmations). System 10 with or without the user may assess the relevancy of risk parameters based on the medical information and previous user-system interactions.

Risk parameters that may be relevant to an individual may have known dependencies with risk models. The dependency relationship may signify that the risk parameter's value is an input for the risk model when run. In some embodiments, only relevant risk models are run. System 10 aids the user in determining which risk models should be run (e.g., which risk models are relevant) by removing dependencies, risk parameters, or risk models that are no longer potentially relevant.

Traditional systems may at times not run relevant risk models because the relationship between a risk parameter and that risk model may not be known. Alternatively, traditional systems may at times run too many risk models, including irrelevant risk models. The more relevant the risk model is the more reliable may be its outcome. System 10 simplifies the computational burden (in the event when too many risk models are used), improves the reliability of risk model outcomes (by running only relevant risk models), and thus provides desired outcome (e.g., the predicted adverse event) faster and more reliably than with traditional systems. Another aspect of the present disclosure is therefore to assist users of system 10 to integrate the outcomes of multiple potentially inter-related risk parameters and risk models.

If, for example, an individual (e.g., a patient) has a risk parameter of being of the male gender, then the risk model for estimating a pregnancy outcome or premature birth is irrelevant, and it would create inefficiencies in decisional processes, e.g., if the decision had to consider a parameter that is clearly known to be irrelevant. Rather, the medical worker may be more interested in using another risk model, e.g., for determining an increased risk towards having prostate cancer. The more risk parameters are confirmed and the more irrelevant risk models are removed then the smaller the set of risk models deemed relevant for the user with respect to an individual under care or medical analysis.

In some instances, risk models may be obtainable, e.g., from published medical literature. Risk models may be used to estimate, compute, and/or predict an individual's risk for a certain adverse event (e.g., a particular health condition, trauma, or other event) based on risk parameters that relate to the individual. Risk models may identify risk parameters that contribute to (or help avoid) the adverse event. Risk models may generate an amount of (e.g., a percentage or probability) risk for the adverse event.

Risk parameters or risk model information is presented in clinical work environments. Risk models may be used at the point of care to plan or conduct a medical procedure. A risk model may be, in some embodiments, a mathematical function that takes as input one or more risk parameters and returns a risk assessment.

Certain risk parameters, when confirmed by a user to a value (e.g., “yes” or “no”), render risk models irrelevant. In some embodiments, when risk models are deemed irrelevant those risk models may not be needed to compute outcomes. Risk parameters, independent of being determined relevant, may be shared between different risk models. In some embodiments, more than one risk model may be interrelated and used to compute outcomes to plan or conduct a medical procedure. In some embodiments, the risk models will consider eligibility criteria, e.g., the eligibility for clinical trial of a medical procedure, and tailored recommendations.

Medical workers may need certain information, for example, based on the type of activity performed by the medical worker or based on a medical specialty (e.g., radiology, cardiology, or other specialty) or diseased body part (e.g., abdomen, heart, or other part or organ). System 10 may filter out risk parameters and risk models that are not relevant or valuable for the medical worker. When some risk parameters are confirmed or when some risk models are run, the outcome of other risk models may be irrelevant. For instance, if an individual is on dialysis or the outcome of a risk model is to put the individual on dialysis, then contrast-induced nephropathy (CIN) would not be relevant, when the patient undergoes percutaneous coronary intervention (PCI). Therefore, if the patient has a confirmed status value that confirms the risk parameter of having end-stage renal disease, then for this patient there would be no value in generating an adverse event prediction based on an Acute Kidney Injury (AKI) risk model that estimates CIN risk or in confirming any risk parameters that exclusively drive those irrelevant risk models.

In some embodiments, system 10 comprises one or more computing devices 18, one or more processors 20, electronic storage 22, external resources 24, and/or other components. Computing devices 18 are configured to provide an interface between users and system 10. Computing devices 18 are configured to provide information to and/or receive information from one or more users. Computing devices 18 include a user interface and/or other components. The user interface may be and/or include a graphical user interface configured to present views and/or fields configured to receive entry and/or selection with respect to risk parameters (or their values), risk models, or other items, and/or provide and/or receive other information. In some embodiments, the user interface includes a plurality of separate interfaces associated with a plurality of computing devices 18, processors 20, and/or other components of system 10.

In some embodiments, one or more computing devices 18 are configured to provide a user interface, processing capabilities, databases, and/or electronic storage to system 10. As such, computing devices 18 may include processors 20, electronic storage 22, external resources 24, and/or other components of system 10. In some embodiments, computing devices 18 are connected to a network (e.g., the internet). In some embodiments, computing devices 18 do not include processor 20, electronic storage 22, external resources 24, and/or other components of system 10, but instead communicate with these components via the network. The connection to the network may be wireless or wired. In some embodiments, computing devices 18 are laptops, desktop computers, smartphones, tablet computers, and/or other computing devices.

Examples of interface devices suitable for inclusion in the user interface include a touch screen, a keypad, touch sensitive and/or physical buttons, switches, a keyboard, knobs, levers, a display, speakers, a microphone, an indicator light, an audible alarm, a printer, and/or other interface devices. The present disclosure also contemplates that computing devices 18 include a removable storage interface. In this example, information may be loaded into computing devices 18 from removable storage (e.g., a smart card, a flash drive, a removable disk) that enables users to customize the implementation of computing devices 18. Other exemplary input devices and techniques adapted for use with computing devices 18 and/or the user interface include, but are not limited to, an RS-232 port, RF link, an IR link, a modem (telephone, cable, etc.) and/or other devices.

Processor 20 is configured to provide information processing capabilities in system 10. As such, processor 20 may comprise one or more of a digital processor, an analog processor, a digital circuit designed to process information, an analog circuit designed to process information, a state machine, and/or other mechanisms for electronically processing information. Although processor 20 is shown in FIG. 1 as a single entity, this is for illustrative purposes only. In some embodiments, processor 20 may comprise a plurality of processing units. These processing units may be physically located within the same device (e.g., a server), or processor 20 may represent processing functionality of a plurality of devices operating in coordination (e.g., one or more servers, computing devices 18, devices that are part of external resources 24, electronic storage 22, and/or other devices.)

In some embodiments, processor 20, external resources 24, computing devices 18, electronic storage 22, and/or other components may be operatively linked via one or more electronic communication links. For example, such electronic communication links may be established, at least in part, via a network such as the Internet, and/or other networks. It will be appreciated that this is not intended to be limiting, and that the scope of this disclosure includes embodiments in which these components may be operatively linked via some other communication media. In some embodiments, processor 20 is configured to communicate with external resources 24, computing devices 18, electronic storage 22, and/or other components according to a client/server architecture, a peer-to-peer architecture, and/or other architectures.

As shown in FIG. 1, processor 20 is configured via machine-readable instructions to execute one or more computer program components. The computer program components may comprise one or more of a risk model management component 30, a risk dependency component 32, a health record management component 34, a user interface component 36, health prediction component 38, and/or other components. Processor 20 may be configured to execute components 30, 32, 34, 36, and/or 38 by software; hardware; firmware; some combination of software, hardware, and/or firmware; and/or other mechanisms for configuring processing capabilities on processor 20.

It should be appreciated that although components 30, 32, 34, 36, and 38 are illustrated in FIG. 1 as being co-located within a single processing unit, in embodiments in which processor 20 comprises multiple processing units, one or more of components 30, 32, 34, 36, and/or 38 may be located remotely from the other components. The description of the functionality provided by the different components 30, 32, 34, 36, and/or 38 described below is for illustrative purposes, and is not intended to be limiting, as any of components 30, 32, 34, 36, and/or 38 may provide more or less functionality than is described. For example, one or more of components 30, 32, 34, 36, and/or 38 may be eliminated, and some or all of its functionality may be provided by other components 30, 32, 34, 36, and/or 38. As another example, processor 20 may be configured to execute one or more additional components that may perform some or all of the functionality attributed below to one of components 30, 32, 34, 36, and/or 38.

In some embodiments, health record management component 34 may extract (e.g., by mining the information) health data from medical information for the sake of predicting risk parameters. As an example, health record management component 34 may search the medical information for condition-specific health data. In some embodiments, health record management component 34 may derive health data using a background ontology. For example, there may be different types of health data grouped and ordered among other types of health data for an individual.

Health record management component 34 may determine risk parameters by extracting information from multiple different types of information items (e.g., documents, reports, charts, graphs, or other information items) of medical information. For example, health record management component 34 may extract health data from (i) a problem list of medical codes/identifiers that encode a clinical condition (e.g., for which the risk parameter is being determined), (ii) laboratory values, including those in some instances that are relative to predetermined thresholds (e.g., systolic positive airway pressure (PAP) being greater than 60 mmHG (millimeter of mercury)), (iii) a medication list or lists of dietary supplements or prescription drugs used to treat a clinical condition, (iv) narrative reports using pattern recognition or more advanced natural language processing that detects un-negated occurrences of the clinical conditions and their normal lexical variants (e.g., “diabetes” and “diabetic”) in particular sections of narrative documents, or (v) other approaches. Health data may therefore take several different forms, such as a piece of text in a narrative report or a code from the background ontology. Health record management component 34 may, in some embodiments, include extraction modules that parse different types of medical information. For example, one extraction module could parse medications and a second one could parse laboratory results. Health data extracted from health record management component 34 may serve as inputs to health prediction component 38.

Health record management component 34 may output health data from which health prediction component 38 may apply thresholds (e.g., based on medical knowledge, user-configuration, or other factors). Health record management component 34 may perform the extraction of health data and determine a risk parameter from the extracted data. In some embodiments, health record management component 34 generates the risk parameter such that its confirmable status value is normalized, e.g., as “yes” or “no.” For example, the user could confirm that Systolic PAP is greater than 60 mmHG (e.g., by the user clicking to confirm a “yes,” a “no,” or another value).

Health record management component 34 may generate risk parameters from known risk parameters using background knowledge and standard definitions in the field. For instance, a user may follow a rule that if the hypertension risk parameter is confirmed to a status value of “yes,” then the hypotension risk parameter may be confirmed to a status value of “no” or “irrelevant.” FIGS. 2A-2C illustrate such risk parameters, i.e., risk parameters with their corresponding status value for an exemplary individual. The corresponding Status may be confirmed to one of a set of values other than “yes” and “no.” For example, a selected status value may be in a numeric range, in an alphanumeric grading scale, or from another set of values, such as “normal,” “moderate,” and “severe.”

In some embodiments, health record management component 34 may leverage the hierarchical or network-like relationships in background ontologies to derive new health data from the extracted health data. For instance, health record management component 34 may leverage health data embedded in an ontology, such as SNOMED clinical terms, radiology lexicon (RadLex), logical observation identifiers names and codes (LOINC), current procedural terminology (CPT), or international classification of diseases (ICD). In one embodiment, health record management component 34 may include an additional mapping operation in converting health data into an ontology. Ontologies typically have interrelationships that have a predetermined meaning, such as “is-a” and “part-of” Therefore, in this embodiment, when health data is extracted by health record management component 34, other health data, which is more general to the extracted health data, may be derived by traversing the “is-a” relationship iteratively. Health record management component 34 may similarly extract codes (e.g., in the ICD) to then derive other, more general codes. Health prediction component 38 may leverage health data thus collected when predicting risk parameters.

In some embodiments, health prediction component 38 may predict risk parameters of an individual based on the obtained medical information (e.g., EMR). Some embodiments support user-system interactions that participate in the prediction of relevant risk parameters. For example, a medical work may know that health data A, B, C, and D are indicative of a diabetes risk parameter, but the medical worker may still be needed because an individual could be diabetic even if health data C and D do not pertain to the individual.

Prediction of a risk parameter may form part of the operation of synthesizing a risk parameter, which includes the operation of confirming a status value of the predicted risk parameter. Health prediction component 38 may use health data extracted from medical information and determine relationships between that data and potential risk parameters. In one example, a risk parameter of diabetes mellitus may be predicted based on a disease identifier (e.g., an ICD code) that indicates diabetes mellitus, a medication list that is indicative of diabetes mellitus (e.g., active insulin use), or an individual with laboratory results from a blood test (e.g., with a glucose level greater than 200 milligrams (mg)/decilitre (dL)). Health prediction component 38 may obtain one or more of these health data points derived from the medical information (by, e.g., health record management component 34) and predict particular risk parameters. In some embodiments, a user of system 10 may need to confirm the prediction for it be considered synthesized, but in other embodiments certain predictions may be of sufficient certainty that a user does not need to make a confirmation.

In one embodiment, health prediction component 38 may use clinically contextual information. In some embodiments, health prediction component 38 may place a weighting factor on candidate risk parameters when selecting from among them to make the prediction. Such a weighting factor may be placed on a demographic (e.g., on an individual belonging to a certain age group for a risk parameter of Alzheimer's, and in other examples, on a race, zip code, economic status, gender, or other demographic, and this may be indicative of a particular risk parameter, especially when weighted more heavily than other risk parameters. Weighted risk parameters enable health prediction component 38 to more reliably predict risk parameters such that a user is more probable or likely to confirm a predicted risk parameter and thus synthesize the risk parameter with respect to an individual.

In some embodiments, health prediction component 38 may include a threshold level. In one embodiment, a threshold may be applied to automatically confirm risk parameters whose certainty value predicted by health prediction component 38 exceeds it. That is, a user interface of system 10 may display to a user a list of confirmable risk parameters that have a probability higher than a given threshold. When the threshold level is crossed, health prediction component 38 may automatically confirm that risk parameter's status value. In these embodiments, the next most likely risk parameters to be relevant may be presented to the user for confirmation in a more efficient manner (e.g., by automatically confirming one or more obviously relevant or irrelevant risk parameters) and thus without requiring user-system interaction in some instances (e.g., when the health data extracted from the medical information makes a strong case for a given risk parameter). In other instances, the extracted health data may be insufficient for automatic confirmation of a risk parameter. In another embodiment, with predetermined thresholds, the risk parameter may be set to a suggested status value, thus enabling more rapid confirmation by the user.

Health prediction component 38 may, in some embodiments, output a status value in a range from 0 to 1 for each risk parameter, whereupon color coding may be used. For example, one or more predicted risk parameters presented to a user for confirmation may be colored red to highlight that that risk parameter has a high likelihood of being confirmed, colored for another reason (e.g., the risk parameter has many risk model dependencies), or colored to signify another characteristic of the predicted risk parameter.

Health prediction component 38 may include decision logic that uses medical information from multiple information sources, including in instances when the medical information is incomplete or with discrepancies. Health data contained in the medical information may therefore be inconsistent (e.g., a certain parameter mentioned in one medical document may be absent in another). For instance, an individual may not have had his diagnostic blood drawn at the same institute as where the individual is currently receiving care, or the physician who prescribed insulin may not have added the diabetes code to the individual's problem list. As a consequence, simple decision rules may not be appropriate for synthesizing risk parameters from extracted health data, e.g., when those risk parameters need to be confirmed by a medical worker.

In some embodiments, the predicted set of risk parameters may be ranked, serialized, or ordered, for ease of review by the user, based on their predicted relevancy (e.g., with the risk parameters predicted most likely to be relevant in a prominent position of a view on a user interface of system 10). The prediction may therefore, in some instances, include filtering and prioritization of the potentially relevant risk parameters. In some embodiments, health prediction component 38 may present candidate risk parameters to a user, for confirmation that the risk parameters are relevant. In some embodiments, one or more of the candidate risk parameters may be ranked, e.g., by a probability that the risk parameter will be confirmed to be relevant by the user. In some embodiments, the list of risk parameters may be displayed to the user in a ranked order and additionally or alternatively in an unranked order.

Some risk parameters may be time dependent, e.g., requiring that a user confirm the risk parameter within a certain time frame. For example, some lab results may only remain valid for a certain period of time (e.g., 30 days) and, consequently, risk parameters confirmed based on the lab value outside of that time period may actually be considered unconfirmed. In another context, a risk parameter may be confirmed by confirming a related risk parameter. That is, in some embodiments, health prediction component 38 may make a prediction based on prior user interactions at system 10 (e.g., with user interface component 36). For instance, health prediction component 38 may suggest (e.g., emphasize or rank) or confirm the diabetes risk parameter as a result of confirming the risk parameter of having consistent glucose level>140 mg/dL twice in a pre-determined period of time. In one embodiment, health record management component 34 may therefore synthesize risk parameters based on previously confirmed risk parameters. In some embodiments, health prediction component 38 may aggregate a plurality of predicted risk parameters and as an aggregate predict another, encompassing risk parameter.

The medical worker may confirm risk parameters at different times and have different credentials when confirming risk parameters. That is, in some embodiments, a date of confirmation and user credentials may both be used to confirm the status value of a predicted risk parameter. For instance, for certain risk parameters a nurse may be capable of confirming the risk parameter but other risk parameters may only be confirmed by an MD.

In some embodiments, health prediction component 38 may learn for each risk parameter a predictive model that takes as input the extraction(s) outputted from health record management component 34. In some instances, the output is labelled with its source to differentiate between more and less reliable data sources. In these instances, a profile of the source document extractor or editor may be included to help differentiate between data entered by senior medical doctors (MDs) versus junior technicians, for instance.

Traditional techniques for synthesizing a risk parameter may be time consuming (e.g., the task may not be straightforward) and in traditional implementations there may be no control fields for editing the risk parameter. Additionally or instead of synthesizing a risk parameter and ranking synthesized risk parameters in order of relevancy, the user may synthesize the risk parameter or rank the synthesized risk parameters based on information from prior user-system interaction (e.g., prior confirmation).

Machine learning techniques known in the field are therefore contemplated herein, and they may include logistic regression, neural network, and rule-learning approaches. In some embodiments, health prediction component 38 may apply (e.g., periodically) the machine learning techniques in predicting risk parameters. In some embodiments, health prediction component 38 may consider a risk parameter as relevant based on predetermined, algorithmically determined, heuristically determined, or user-configurable rules. For example, health prediction component 38 may apply, in some embodiments, Boolean logic to generate a suggested status for the risk parameter based on the extracted output, e.g., from health record management component 34. For instance, health prediction component 38 may synthesize as “yes” a status value for a diabetes risk parameter, if an ICD code of “10” were extracted.

In some embodiments, user interface component 36 may provide a user interface of system 10 (e.g., pertaining to computing device 18) that allows the user to select a status value of a risk parameter from status values predicted by health prediction component 38. User interface component 36 may then store (e.g., in electronic storage 22 or with external resources 24) this user-system interaction (e.g., a confirmation).

The database may store all values extracted by health record management component 34, predicted risk parameters, and status values of risk parameters confirmed at the user interface. For instance, the database may store a user confirming or not an individual having diabetes even though there may be a diabetes code on an individual's problem list. A database of electronic storage 22 or external resources 24 may additionally, in some embodiments, store a time stamp or a user's credential information. The database may additionally or alternatively store contextual information (e.g., clinical context of an individual). The database may additionally or alternatively store user profile information (e.g., role and rank), such as, for instance, an MD, fellow, nurse, technician, biller, etc.

User interface component 36 may display, in some embodiments, an interactive user interface for reviewing and confirming risk parameters. In some embodiments, user interface component 36 may alert the user when the extracted health data and prior user-system interaction indicate that a predicted risk parameter should be confirmed. The user may also independently indicate that he or she desires to determine a status value of a risk parameter. User interface component 36 may therefore display the predicted risk parameters on the user interface, as shown in FIGS. 2A-2C, and when clicked the user interface may display available status values for that predicted risk parameter.

In one embodiment, user interface component 36 may provide the user with a field to search for candidate risk parameters using, e.g., key words. For instance, the user may search the individual's medical information, specifically in the problem list of active diagnoses for diabetes-related codes or the medications list for insulin. If either is found, user interface component 36 may bring this to the user's attention, thus assisting the user to efficiently confirm particular risk parameters.

In some embodiments, user interface component 36 may support a user interface that displays risk score information, e.g., after running a risk model. Risk model management component 30 may collaborate with health prediction component 38 to know which risk models to run. One or more risk parameters may therefore be highlighted in a display to the user, e.g., in a tabular view of exemplary risk parameters (with their corresponding status values), as shown in FIGS. 2A, 2B, and 2C. The highlighting of a predicted risk parameter encourages the user to confirm it. And when one risk parameter is confirmed others may be automatically confirmed. The highlighting may therefore help expedite confirmation of all risk parameters driving risk models that are considered contextually relevant by risk dependency component 32. For instance, if risk dependency component 32 considers that the AKI model should be run, all risk parameters that have not been confirmed driving this risk model will be emphasized (e.g., highlighted). Similarly, in another embodiment, one or more risk parameters may be deemphasized if they only drive risk models that are rendered irrelevant.

FIG. 2B shows risk parameter 46 (end-stage renal disease) as visually highlighted, along with “click to confirm” button 48. But any emphasis technique is contemplated (e.g., when a risk parameter is emphasized it could be at the top of the list in the table or it could be emphasized as bold, italics, underlined, all capital letters, or via another emphasis technique). FIG. 2C shows the Status of risk parameter 46 as confirmed to a “yes” status value. As a consequence, the risk parameters hypertension, anemia, chronic heart failure, diabetes, age>75 years, and creatinine are confirmed (e.g., automatically) as irrelevant. The user is then encouraged to confirm the Status for risk parameter 50 (hypotension). In some embodiments, user interface component 36 may automatically deemphasize at the user interface all risk parameters that are inputs to a risk model rendered irrelevant.

In some embodiments, risk model management component 30 is configured to manage risk parameters, risk models, their relationships with one another, and other aspects related to the risk parameters or risk models. In some embodiments, risk dependency component 32 may be configured, among other operations, to facilitate identification of risk parameters or models that are relevant with respect to an individual or identification of risk parameters or risk models that are irrelevant with respect to the individual.

A risk model may comprise a function that takes as input values of one or more risk parameters and provides an assessment as output (e.g., a prediction of an adverse event, a health risk assessment for an individual, an eligibility assessment for one or more treatments for the individual, a recommendation assessment for the individual, or other assessment). Risk model management component 30 may use confirmed risk parameter information and, in some embodiments, outcomes of other risk models (e.g., a score) when running.

Risk model management component 30 may flag risk models as irrelevant based on the confirmed risk parameter. In one implementation, a set of rules is used to determine which risk models are irrelevant. The rules may be based on a Boolean combination of risk parameters and where appropriate the outcomes of other risk models to then indicate whether one or more risk models are relevant. For example, if end-stage renal disease is confirmed as a relevant risk parameter, then the AKI risk model may be rendered irrelevant.

In some embodiments, risk model management component 30 may compute risk scores based on risk parameters. Risk model management component 30 may have access to a risk model database (e.g., of electronic storage 22 or external resources 24). The database may contain all risk scores, their input risk parameters, a relevance status, and other aspects. In some embodiments, risk model management component 30 may transform clinical context (e.g., as input from a user or derived from medical information pertaining to the individual under medical care) into a set of one or more relevant risk models.

Risk model management component 30 may maintain a mapping between contextual settings (e.g., a PCI patient or echo interpretation workflow of an end-stage renal patient) and relevant risk models. In one embodiment, the contextual setting may be arrived at by filtering out context not related to a profile of the user (e.g., of an interventional cardiologist or echo cardiologist). In another embodiment, the user may select the context from a drop-down menu of a user interface. In some embodiments, risk model management component 30 may identify risk models from the risk model database that are relevant whenever a context is known or has been selected or changed.

In some embodiments, risk model management component 30 may manage interactions between risk scores. In some embodiments, risk model management component 30 may have access to a risk parameter persistence store (e.g., of electronic storage 22 or external resources 24) that persists individual-specific risk parameters and user-system interaction data. Risk model management component 30 may retrieve previously confirmed risk parameter values from the risk parameter persistence store. This database may maintain risk parameters that have been established for patients previously. For instance, the database may maintain for every confirmed risk parameter particular circumstances, e.g., who confirmed it (and for which individual), the context, and the date of confirmation. The database may be queried for previously stored risk parameter information. In one embodiment, the database is queried based on the context of the individual.

In some embodiments, risk model management component 30 may be configured to generate one or more graphs and store the generated graphs (e.g., in one or more databases of electronic storage 22, one or more databases of external resources 24, or other destinations). In some embodiments, risk model management component 30 is configured to generate a graph comprising nodes and edges, where each edge links two of the nodes, and where the nodes comprise nodes of the first node type that respectively correspond to risk parameters, nodes of a second node type that respectively correspond to risk models, or other nodes of other node types. In one use case, each of the first-type nodes may represent a risk factor, a risk marker, clinical condition, or other risk parameter, and each of the second-type nodes may represent a risk model. In another use case, each of the risk models that represent one of the second-type nodes may be configured to take one or more values of the risk parameters as input to estimate the likelihood that an individual has one or more health conditions, estimate the likelihood that an individual is at risk of having one or more health conditions, or provide other outputs. In some embodiments, risk model management component 30 is configured to generate the graph such that an edge links a given first-type node of the graph to a given second-type node of the graph based on a risk model of the given second-type node being configured to take a value of a risk parameter of the given first-type node as input (e.g., to estimate a likelihood that an individual has or is at risk of having one or more health conditions).

In some embodiments, risk model management component 30 is configured to obtain the graph from one or more databases or other sources. In some embodiments, risk dependency component 32 is configured to process the obtained graph to generate a resulting graph with respect to a first individual. As an example, risk dependency component 32 may generate the resulting graph by assessing one or more nodes or edges of the obtained graph and/or modifying the obtained graph on the assessment of the nodes or edges. As a further example, risk dependency component 32 may modify the obtained graph by adding one or more nodes or edges to the obtained graph, removing one or more nodes or edges from the obtained graph, modifying one or more aspects of nodes or edges of the obtained graph, or performing other modifications.

In some embodiments, upon generating the resulting graph, risk dependency component 32 is configured to select one or more risk models based on the resulting graph that are to be used to perform analysis of at least one health condition of the first individual. In some embodiments, risk dependency component 32 is configured to select the risk models from a set of risk models corresponding to one or more second-type nodes of the resulting graph that respectively have at least one edge linking the respective second-type node to at least one first-type node of the resulting graph.

Risk dependency component 32 may determine dependencies between risk parameters and risk models using, e.g., a table of risk parameters with links to a table of risk models. That is, in some embodiments, a user of system 10 may with risk dependency component 32 configure rules (e.g., in a table) such that certain risk parameters render certain risk models irrelevant.

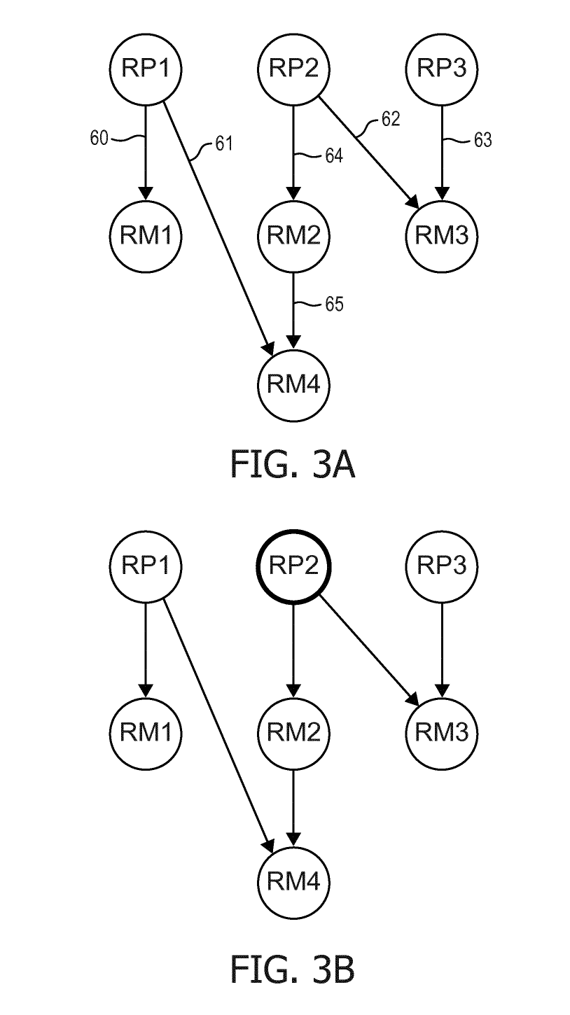

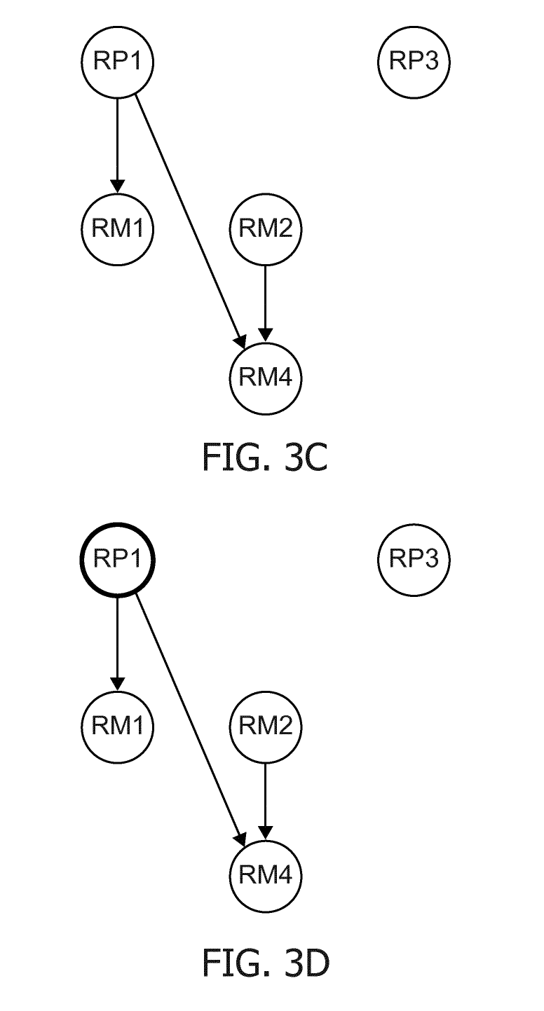

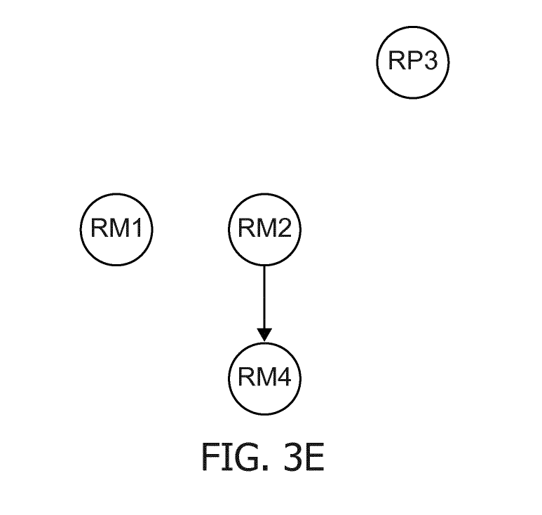

FIGS. 3A, 3B, 3C, 3D, and 3E illustrate examples of risk model nodes (second node type) in a directed graph connected via edges to risk parameter nodes (first node type), which should be confirmed for the sake of removing irrelevant risk models, in accordance with one or more embodiments. In some embodiments, risk dependency component 32 may arrange a set of rules as the directed graph, where each directed edge may indicate whether the status value of one risk parameter or outcome of one risk model renders another risk model irrelevant. For example, one edge may indicate that if the risk parameter is confirmed then the risk model is relevant, but another edge may indicate that if the risk parameter is confirmed then the risk model is irrelevant.

FIG. 3A is a graph depicting three risk parameter (RP) nodes and four risk model (RM) nodes. Nodes RP1, RP2, and RP3 are of the first type, nodes RM1, RM2, RM3, and RM4 are of the second type, and edges 60, 61, 62, 63, 64, and 65 link two nodes, as illustrated in this graph. An edge indicates that there is an outcome of one node that may render the other node irrelevant. For instance, there may be a state of node RP2 that renders node RM2 irrelevant and there may be a state of node RP2 that renders node RM3 irrelevant.

In some embodiments, risk dependency component 32 is configured to determine one of the first-type nodes of the obtained graph as a node to be assessed. In some embodiments, risk dependency component 32 is configured to select the determined first-type node (as the node to be assessed) based on a number of edges that link the first-type node to a given second-type node. As an example, the first-type node may be selected based on a determination that the first-type node has more such edges than other first-type nodes of the obtained graph (e.g., the selected first-type node has the most edges linking to a given second-type node as compared to all other first-type nodes in a set of first-type nodes). As another example, the first-type node may be selected based on a determination that the first-type node has less such edges than other first-type nodes of the first obtained graph.

After obtaining a graph with all potentially relevant risk parameters and risk models, including their interdependencies, risk dependency component 32 may, in some embodiments, begin to select the risk models that will be run by first identifying a risk parameter that has the most edges to risk models that it may render irrelevant. In the example of FIG. 3B, node RP2 is thus identified but, in some instances, risk dependency component 32 may identify instead (or identify in either order) node RP1. This is because nodes RP1 and RP2 both have the most edges (two) that could render risk models irrelevant; in this example, nodes RP1 and RP2 may render nodes RM1, RM2, RM3, and RM4 irrelevant, respectively.

In this fashion, risk dependency component 32 may interoperate with health prediction component 38, since health prediction component 38 may be promoted to emphasize or place at the top of a candidate list (e.g., first row of a table, as shown in FIG. 2A) contents of node RP2. Upon confirming the status value of node RP2 (e.g., confirmed to a status value of “no”), risk dependency component 32 may render node RM3 irrelevant by removing it from the graph, as illustrated in FIGS. 3C-3E. In this example, confirming node RP2 renders node RM2 relevant, which is why risk dependency component 32 does not remove it from the graph.

In the example illustrated with FIGS. 3B and 3C, although edges 64 and 62 have both been removed, this is merely an implementation specific detail and different approaches are contemplated. For instance, after a risk parameter is confirmed, risk dependency component 32 may only remove the edges and nodes that render risk models irrelevant. Similarly, in some implementations, a confirmed risk parameter (e.g., node RP2) may be removed (as shown in FIGS. 3C-3E), but in other implementations node RP2 may remain in the graph. In another embodiment, one or more risk parameters or edges may be hidden or otherwise displayed according to their relevance (e.g., with colors or an intermittent line).

In some embodiments, risk dependency component 32 may be configured to confirm a value of a risk parameter of the first-type node (e.g., selected to be assessed) with respect to the first individual. In some embodiments, based on the risk parameters confirmed by the user to be relevant, risk dependency component 32 may flag as irrelevant one or more other risk parameters, e.g., the ones that have not been confirmed by the user. Risk dependency component 32 also may utilize a dependency between determined risk parameters and known risk models.

In some embodiments, risk dependency component 32 is configured to perform the removal of one or more edges linking second-types node to one or more first-type nodes (e.g., including the edge linking the first-type node and the second-type node) from the obtained graph based on the value of the risk parameter of the first-type node. In some embodiments, removal of an edge or node from the obtained graph comprises deleting the edge or node from the obtained graph. In some embodiments, removal of an edge or node from the obtained graph comprises labeling the edge or node with a value indicating that the edge or node is not to be considered when selecting a risk model to be used for performing analysis with respect to the first individual.

In some embodiments, risk dependency component 32 may update the directed graph of nodes and edges, when a risk parameter is confirmed or the outcome of another risk model is computed. The graph may be updated by removing edges or nodes that will not render other risk models irrelevant. For instance, if the end-stage renal disease risk parameter is set (e.g., to “no,” “false,” or other setting), then this risk parameter may be removed from the graph as well as all of its edges to risk models where such an edge if set differently (e.g., “yes,” “true,” or other different setting) would have rendered those risk models irrelevant.

In some embodiments, risk dependency component 32 is configured to determine whether a risk model of the second-type node satisfies a relevance threshold based on the value of the risk parameter of the first-type node. In some embodiments, risk dependency component 32 is configured to remove one or more edges linking the second-type node to one or more first-type nodes (e.g., including the edge linking the first-type node and the second-type node) responsive to a determination that the risk model of the second-type node fails to satisfy the relevance threshold.

In some embodiments, risk dependency component 32 may be configured to remove the second-type node from the obtained graph based on the value of the risk parameter of the first-type node (to which, prior to the removal, the second-type node shares an edge). In some embodiments, risk dependency component 32 is configured to remove the second-type node from the obtained graph responsive to a determination that the risk model of the second-type node fails to satisfy the relevance threshold. As an example the determination of whether the risk model of the second-type node fails to satisfy the relevance threshold may be based on the value of the risk parameter of the first-type node.

In some embodiments, risk dependency component 32 is configured to remove one or more other nodes from the obtained graph based on a respective number of edges of the other nodes. In some embodiments, risk dependency component 32 is configured to remove one or more other first-type nodes from the obtained graph based on respective numbers of edges of the other first-type nodes that link the other first-type nodes to a given second-type node (e.g., to any second-type node remaining in the graph). As an example, the processing of the obtained graph (during which one or more edges or nodes is removed) may cause one or more nodes of the graph to have no edges that link those nodes to certain types of nodes. In one use case, for example, if a given first-type node (representing a risk parameter) no longer has an edge linking the given first-type node to any second-type node (representing a risk model) after the removal of one or more second-type nodes (to which the given first-type node used to be linked), then the given first-type node (and/or its risk parameter) may be deemed irrelevant and may be removed from the obtained graph. In this way, risk parameters that are no longer relevant to any risk models represented in the resulting graph may be removed, for example, to avoid a need to consider such risk parameters when performing analysis of an individual's health conditions based on the resulting graph.

In some embodiments, risk dependency component 32 may add to a candidate list any risk parameters that may render any risk model irrelevant that has not already been rendered irrelevant. If there are multiple candidate risk parameters, then the risk parameter ranked highest is that which has the largest out-degree, i.e., edges which render the most number of risk models irrelevant.

In some embodiments, risk dependency component 32 may consider all confirmed risk parameters to then add to another candidate list any risk model that cannot be rendered irrelevant by the confirmation of any other risk parameter or the outcome of any other risk model, e.g., any risk model that has no incoming edges in the graph from unconfirmed risk parameters. Node RM2 in FIG. 3D is such an example. By this technique, the number of risk models used to predict an adverse event is reduced. This number may be reduced further, in some embodiments, based on selection of a risk model(s) that has a fewest number of edges from unconfirmed risk parameters.

In some embodiments, risk dependency component 32 may identify another risk parameter node that has edges to risk model nodes it can render irrelevant. And thus risk dependency component 32 may iteratively identify risk parameter nodes, optionally emphasize the risk parameter to the user for confirmation (e.g., on a user interface), and then remove edges to risk model nodes rendered irrelevant when the risk parameter is confirmed to a particular status value. For instance, as depicted in FIGS. 3C-3E, risk dependency component 32 may not render either node RM1 or node RM2 irrelevant. Risk dependency component 32 may then determine that three risk models (of nodes RM1, RM2, and RM4) are relevant for running to predict desired information, e.g., information pertaining to a likelihood that the individual has or is at risk of having one or more health conditions or having an adverse event take place. In another example (not shown), confirmation of node RP1 to a different status value may render both nodes RM1 and RM4 irrelevant, leaving the user with only one relevant risk model (i.e., the risk model of node RM2) to run. In this other example, the user has an even less number of risk models to run than before, which improves on run-time for executing risk models to determine the desired information.

Returning to FIG. 1, electronic storage 22 comprises electronic storage media that electronically stores information. The electronic storage media of electronic storage 22 may comprise one or both of system storage that is provided integrally (i.e., substantially non-removable) with system 10 and/or removable storage that is removably connectable to system 10 via, for example, a port (e.g., a USB port, a firewire port, etc.) or a drive (e.g., a disk drive, etc.). Electronic storage 22 may be (in whole or in part) a separate component within system 10, or electronic storage 22 may be provided (in whole or in part) integrally with one or more other components of system 10 (e.g., a computing device 18, processor 20, etc.). In some embodiments, electronic storage 22 may be located in a server together with processor 20, in a server that is part of external resources 24, in computing devices 18, and/or in other locations. Electronic storage 22 may comprise one or more of optically readable storage media (e.g., optical disks, etc.), magnetically readable storage media (e.g., magnetic tape, magnetic hard drive, floppy drive, etc.), electrical charge-based storage media (e.g., EPROM, RAM, etc.), solid-state storage media (e.g., flash drive, etc.), and/or other electronically readable storage media. Electronic storage 22 may store software algorithms, information obtained and/or determined by processor 20, information received via computing devices 18 and/or other external computing systems, information received from external resources 24, and/or other information that enables system 10 to function as described herein.

External resources 24 include sources of information (e.g., databases, websites, etc.), external entities participating with system 10 (e.g., a medical records system of a health care facility that stores patient census information), one or more servers outside of system 10, a network (e.g., the internet), electronic storage, equipment related to Wi-Fi technology, equipment related to Bluetooth® technology, data entry devices, and/or other resources. In some implementations, some or all of the functionality attributed herein to external resources 24 may be provided by resources included in system 10. External resources 24 may be configured to communicate with processor 20, computing device 18, electronic storage 22, and/or other components of system 10 via wired and/or wireless connections, via a network (e.g., a local area network and/or the internet), via cellular technology, via Wi-Fi technology, and/or via other resources.

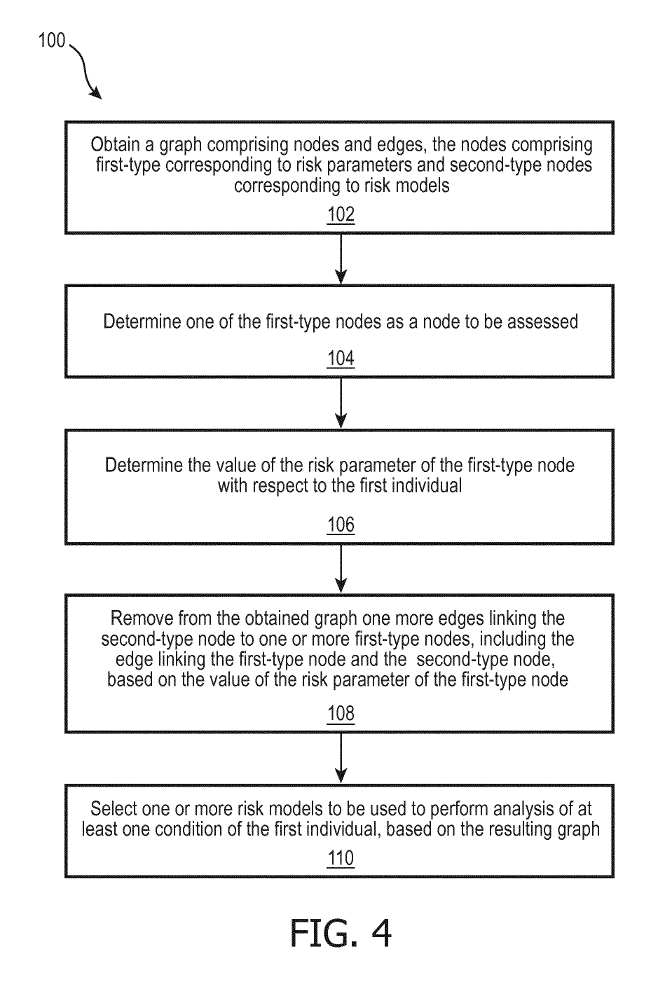

FIG. 4 illustrates a method 100 for facilitating computational analysis of health conditions via graph generation, in accordance with one or more embodiments. Method 100 may be performed with a computer system comprising one or more hardware processors and/or other components. The hardware processors are configured by machine readable instructions to execute computer program components. The operations of method 100 presented below are intended to be illustrative. In some embodiments, method 100 may be accomplished with one or more additional operations not described, and/or without one or more of the operations discussed. Additionally, the order in which the operations of method 100 are illustrated in FIG. 4 and described below is not intended to be limiting.

In some embodiments, method 100 may be implemented in one or more processing devices (e.g., a digital processor, an analog processor, a digital circuit designed to process information, an analog circuit designed to process information, a state machine, and/or other mechanisms for electronically processing information). The processing devices may include one or more devices executing some or all of the operations of method 100 in response to instructions stored electronically on an electronic storage medium. The processing devices may include one or more devices configured through hardware, firmware, and/or software to be specifically designed for execution of one or more of the operations of method 100.

At an operation 102, a graph comprising nodes and edges may be obtained, the nodes comprising first-type nodes corresponding to risk parameters and second-type nodes corresponding to risk models. As an example, the risk parameters may comprise risk factors, risk markers, or other risk parameters. The risk models may be configured to take one or more values of the risk parameters as input to estimate the likelihood that an individual has one or more health conditions, estimate the likelihood that an individual is at risk of having one or more health conditions, or provide other outputs. In some embodiments, operation 102 is performed by a processor component the same as or similar to risk model management component 30 (shown in FIG. 1 and described herein).

At an operation 104, one of the first-type nodes to be assessed may be determined as such. As an example, the first-type node may be selected from the first-type nodes (as a node to be assessed) based on a number of edges that link the first-type node to a given second-type node (e.g., based on the selected first-type node having more such edges than other first-type nodes, the selected first-type node having less such edges than other first-type nodes, or other criteria related to the number of such edges). In some embodiments, operation 104 is performed by a processor component the same as or similar to risk dependency component 32 (shown in FIG. 1 and described herein).

At an operation 106, the value of the risk parameter of the first-type node may be determined with respect to the first individual. In some embodiments, operation 106 is performed by a processor component the same as or similar to risk dependency component 32 (shown in FIG. 1 and described herein).

At an operation 108, one or more edges linking the second-type node to one or more first-type nodes (including the edge linking the first-type node and the second-type node) may be removed from the obtained graph based on the value of the risk parameter of the first-type node. As an example, the removal may be performed by deleting the edges from the obtained graph. As another example, the removal may be performed by labeling the edges with a value indicating that the edges are not to be considered when selecting a risk model (to be used for performing analysis with respect to the first individual). In some embodiments, operation 108 is performed by a processor component the same as or similar to risk dependency component 32 (shown in FIG. 1 and described herein).

At an operation 110, based on the resulting graph, one or more risk models may be selected to be used to perform analysis of at least one health condition of the first individual. As an example, the risk models may be selected from a set of risk models corresponding to one or more second-type nodes of the resulting graph that respectively have at least one edge linking the respective second-type node to at least one first-type node of the resulting graph. In some embodiments, operation 108 is performed by a processor component the same as or similar to risk model management component 30 (shown in FIG. 1 and described herein).

In some embodiments, method 100 further comprises generating, based on the selected risk models, one or more predictions related to least one health condition of the first individual. In some embodiments, the foregoing operation is performed by a processor component the same as or similar to risk model management component 30 (shown in FIG. 1 and described herein).

In some embodiments, method 100 further comprises determining, based on the value of the risk parameter of the first-type node, whether a risk model of the second-type node satisfies a relevance threshold. In some embodiments, the foregoing operation is performed by a processor component the same as or similar to risk dependency component 32 (shown in FIG. 1 and described herein). In some embodiments, with respect to operation 108, the edges linking the second-type node to the first-type nodes may be removed responsive to a determination that the risk model of the second-type node fails to satisfy the relevance threshold.

In some embodiments, method 100 further comprises removing one or more other first-type nodes from the obtained graph based on respective numbers of edges of the other first-type nodes that link the other first-type nodes to a given second-type node. In some embodiments, the foregoing operation is performed by a processor component the same as or similar to risk dependency component 32 (shown in FIG. 1 and described herein).

In some embodiments, method 100 further comprises: determining, subsequent to the removal of the edge linking the first-type node to the second-type node from the obtained graph, another first-type node in the obtained graph that has an edge linking the other first-type node to another second-type node in the obtained graph; determining a value of a risk parameter of the other first-type node with respect to the first individual; and removing one or more edges linking the other second-type node to one or more first-type nodes, including the edge linking the other first-type node and the other second-type node, from the obtained graph based on the value of the risk parameter of the other first-type node. In some embodiments, the foregoing operations are performed by a processor component the same as or similar to risk dependency component 32 (shown in FIG. 1 and described herein).

Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

1. A system configured to facilitate computational analysis of health conditions via graph generation, the system comprising one or more hardware processors configured by machine readable instructions to:
obtain a graph comprising nodes and edges, each of the edges linking two of the nodes and indicating a dependency between the two nodes linked by the edge, the nodes comprising nodes of a first node type that respectively correspond to risk parameters and nodes of a second node type that respectively correspond to risk models, the risk models being configured to take one or more values of the risk parameters as input to estimate a likelihood that an individual has or is at risk of having one or more health conditions;
process the obtained graph to generate a resulting graph for a first individual with reduced dependencies, wherein processing the obtained graph comprises:
determining one of the first-type nodes as a node to be assessed, the first-type node having an edge linking the first-type node to a second-type node in the obtained graph;
determining a value of a risk parameter of the first-type node with respect to the first individual; and
removing one or more edges linking the second-type node to one or more first-type nodes, including the edge linking the first-type node and the second-type node, from the obtained graph based on the value of the risk parameter of the first-type node; and
select, based on the resulting graph with reduced dependencies, one or more risk models to be used to perform analysis of at least one health condition of the first individual such that the one or more risk models are selected from a set of risk models corresponding to one or more second-type nodes of the resulting graph that respectively have at least one edge linking the respective second-type node to at least one first-type node of the resulting graph.

2. The system of claim 1, wherein the obtained graph is configured such that an edge links a given first-type node of the obtained graph to a given second-type node of the obtained graph based on a risk model of the given second-type node being configured to take a value of a risk parameter of the given first-type node as input to estimate a likelihood that an individual has or is at risk of having one or more health conditions.

3. The system of claim 1, wherein the one or more hardware processors are configured to:
determine, based on the value of the risk parameter of the first-type node, whether a risk model of the second-type node satisfies a relevance threshold,
wherein the one or more hardware processors are configured to remove the one or more edges linking the second-type node to the one or more first-type nodes by removing the one or more edges from the obtained edge responsive to a determination that the risk model of the second-type node fails to satisfy the relevance threshold.

4. The system of claim 1, wherein the one or more hardware processors are configured to process the obtained graph by removing the second-type node from the obtained graph based on the value of the risk parameter of the first-type node.

5. The system of claim 4, wherein removal of an edge or node from the obtained graph comprises deleting the edge or node from the obtained graph.

6. The system of claim 4, wherein removal of an edge or node from the obtained graph comprises labeling the edge or node with a value indicating that the edge or node is not to be considered when selecting a risk model to be used for performing analysis with respect to the first individual.

7. The system of claim 1, wherein the one or more hardware processors are configured to process the obtained graph by removing one or more other first-type nodes from the obtained graph based on respective numbers of edges of the one or more other first-type nodes that link the one or more other first-type nodes to a given second-type node.

8. The system of claim 1, wherein the one or more hardware processors are configured to determine the first-type node as the node to be assessed by selecting the first-type node from the first-type nodes based on a number of edges that link the first-type node to a given second-type node.

9. The system of claim 1, wherein the one or more hardware processors are configured to process the obtained graph by:
determining, subsequent to the removal of the edge linking the first-type node to the second-type node from the obtained graph, another first-type node in the obtained graph that has an edge linking the other first-type node to another second-type node in the obtained graph;
determine a value of a risk parameter of the other first-type node with respect to the first individual; and
removing one or more edges linking the other second-type node to one or more first-type nodes, including the edge linking the other first-type node and the other second-type node, from the obtained graph based on the value of the risk parameter of the other first-type node.

10. The system of claim 1, wherein the one or more hardware processors are configured to:
generate, based on the selected one or more risk models, one or more predictions related to at least one health condition of the first individual.

11. A method for facilitating computational analysis of health conditions via graph generation, the method being implemented by one or more hardware processors configured by machine-readable instructions, the method comprising:
obtaining a graph comprising nodes and edges, each of the edges linking two of the nodes and indicating a dependency between the two nodes linked by the edge, the nodes comprising nodes of a first node type that respectively correspond to risk parameters and nodes of a second node type that respectively correspond to risk models, the risk models being configured to take one or more values of the risk parameters as input to estimate a likelihood that an individual has or is at risk of having one or more health conditions;
processing the obtained graph to generate a resulting graph for a first individual with reduced dependencies, wherein processing the obtained graph comprises:
determining one of the first-type nodes as a node to be assessed, the first-type node having an edge linking the first-type node to a second-type node in the obtained graph;
determining a value of a risk parameter of the first-type node with respect to the first individual; and
removing one or more edges linking the second-type node to one or more first-type nodes, including the edge linking the first-type node and the second-type node, from the obtained graph based on the value of the risk parameter of the first-type node; and
selecting, based on the resulting graph with reduced dependencies, one or more risk models to be used to perform analysis of at least one health condition of the first individual such that the one or more risk models are selected from a set of risk models corresponding to one or more second-type nodes of the resulting graph that respectively have at least one edge linking the respective second-type node to at least one first-type node of the resulting graph.

12. The method of claim 11, wherein the obtained graph is configured such that an edge links a given first-type node of the obtained graph to a given second-type node of the obtained graph based on a risk model of the given second-type node being configured to take a value of a risk parameter of the given first-type node as input to estimate a likelihood that an individual has or is at risk of having one or more health conditions.

13. The method of claim 11, further comprising:
determining, based on the value of the risk parameter of the first-type node, whether a risk model of the second-type node satisfies a relevance threshold,
wherein removing the one or more edges linking the second-type node to the one or more first-type nodes comprises removing the one or more edges from the obtained edge responsive to a determination that the risk model of the second-type node fails to satisfy the relevant threshold.

14. The method of claim 11, wherein processing the obtained graph comprises removing the second-type node from the obtained graph based on the value of the risk parameter of the first-type node.

15. The method of claim 11, wherein processing the obtained graph comprises removing one or more other first-type nodes from the obtained graph based on respective numbers of edges of the one or more other first-type nodes that link the one or more other first-type nodes to a given second-type node.

16. (canceled)

17. (canceled)

18. (canceled)

19. (canceled)

20. (canceled)
